(19) United States
(12) Patent Application Publication
(10) Pub. No.: US 2024/0233949 A9
(43) Pub. Date: Jul. 11, 2024

(54) RECOVERY LEVEL ESTIMATION DEVICE, RECOVERY LEVEL ESTIMATION METHOD, AND RECORDING MEDIUM

(71) Applicant: NEC Corporation, Minato-ku, Tokyo (JP)

(72) Inventors: Toshinori HOSOI, Tokyo (JP); Shoji YACHIDA, Tokyo (JP)

(73) Assignee: NEC Corporation, Minato-ku, Tokyo (JP)

(21) Appl. No.: 18/278,959

(22) PCT Filed: Jul. 6, 2021
(86) PCT No.: PCT/JP2021/025427

§ 371 Date: Aug. 25, 2023

(51) Int. Cl. G06T 7/00; A61B 3/00; A61B 3/113; A61B 3/14; G06T 7/246

(52) U.S. Cl. CPC G06T 7/0012; A61B 3/0025; A61B 3/113; A61B 3/14; G06T 7/246; G06T 2207/20081; G06T 2207/30041

(57) ABSTRACT

In a recovery level estimation device, an image acquisition means acquires images in which eyes of a patient are captured. An eye movement feature extraction means extracts an eye movement feature which is a feature of an eye movement based on the images. A recovery level estimation means estimates a recovery level of the patient based on the eye movement feature by using a recovery level estimation model which has been learned by machine learning in advance.

### TECHNICAL FIELD

The present disclosure relates to a technique for estimating a recovery level of a patient.

### BACKGROUND ART

While healthcare costs are putting pressure on national finances worldwide, the number of patients with cerebrovascular diseases in Japan stands at 1,115,000, with annual healthcare costs amounting to more than 1.8 trillion yen. The number of stroke patients is expected to increase as the birthrate declines and the population ages; however, medical resources are limited, and there is a strong need for operational efficiency not only in acute care hospitals but also in convalescent rehabilitation hospitals.

Because cerebral infarction can cause a serious sequela unless emergency transport and measures are taken promptly after onset, it is important to detect and take measures as early as possible while symptoms are mild. Approximately half of the patients with cerebral infarction will develop cerebral infarction again within 10 years and will likely recur the same type of cerebral infarction as the first. Therefore, there is also a strong need for early detection of signs of recurrence.

However, in order to measure a recovery level of a patient in a convalescent rehabilitation hospital, it is necessary for a medical professional to accompany the patient and conduct various tests, which are time-consuming and labor-intensive. Accordingly, the frequency of measuring a recovery level is reduced, feedback to patients and providers will be lost, and patients will be less motivated to rehabilitate, resulting in reduced rehabilitation volume and delayed review of inappropriate rehabilitation plans, which will reduce the effectiveness of recovery. In addition, signs of recurrence are difficult for the patient to recognize on his or her own and often do not occur in time for periodic examinations and medical examinations.

Patent document 1 describes a more objective quantification of recovery status related to gait, based on a movement of a patient and eye movements while walking. Patent document 2 describes the estimation of psychological states from features based on eye movements. Patent document 3 describes determining reflexivity of the eye movements under predetermined conditions. Patent document 4 describes estimating a recovery transition based on movement information quantified from data of a rehabilitation subject.

### PRECEDING TECHNICAL REFERENCES

Patent Document
Patent Document 1: Japanese Laid-open Patent Publication No. 2019-067177
Patent Document 2: Japanese Laid-open Patent Publication No. 2017-202047
Patent Document 3: Japanese Laid-open Patent Publication No. 2020-000266
Patent Document 4: International Publication Pamphlet No. WO20/008657

### SUMMARY

### Problem to be Solved by the Invention

Traditionally, estimation of a recovery level of a patient has been conducted by quantifying a recovery status by having a medical professional or a specialist visually or palpatively evaluate the patient performing a given operation. It is also known to quantify a recovery status of the patient in a remote location by transmitting a video of movements of the patient and a human body posture analysis result as data, and allowing the medical professional or the specialist to visually evaluate the data. In addition, Patent Document 1 describes a medical information processing system which quantifies a recovery status by analyzing a manner in which a human body moves based on a video of a walking scene of the patient.

In order to estimate the recovery level using a traditional method, the patient needs to go to a hospital where the medical personnel and the specialist are available. However, many patients have difficulty going to the hospital for a variety of reasons. By transmitting patient data, hospital Bits of the patient are reduced, but it requires a lot of time and effort on the medical staff and other professionals to visually evaluate the patient data. Moreover, a method of quantifying recovery status based on the video of the walking scene does not require much effort on the medical personnel and the like, but it can only evaluate the patient who has recovered to a level where the patient can walk, and there is also the problem of a risk of falling when walking.

It is one object of the present disclosure to quantitatively estimate the recovery level without burdening the patient or the medical professional.

### Means for Solving the Problem

According to an example aspect of the present disclosure, there is provided a recovery level estimation device including:

an image acquisition means configured to acquire images in which eyes of a patient are captured;

an eye movement feature extraction means configured to extract an eye movement feature which is a feature of an eye movement based on the images; and

a recovery level estimation means configured to estimate a recovery level of the patient based on the eye movement feature by using a recovery level estimation model which has been learned by machine learning in advance.

According to another example aspect of the present disclosure, there is provided a method including:

acquiring images in which eyes of a patient are captured;

extracting an eye movement feature which is a feature of an eye movement based on the images; and

estimating a recovery level of the patient based on the eye movement feature by using a recovery level estimation model which has been learned by machine learning in advance.

According to a further example aspect of the present disclosure, there is provided a recording medium storing a program, the program causing a computer to perform a process including:

acquiring images in which eyes of a patient are captured;

extracting an eye movement feature which is a feature of an eye movement based on the images; and

estimating a recovery level of the patient based on the eye movement feature by using a recovery level estimation model which has been learned by machine learning in advance.

### Effect of the Invention

According to the present disclosure, it becomes possible to quantitatively estimate a recovery level without burdening a patient or a medical professional,

### BRIEF DESCRIPTION OF THE DRAWINGS

FIG. 1 illustrates a schematic configuration of a recovery level estimation device.

FIG. 2 illustrates a hardware configuration of the recovery level estimation device.

FIG. 3 illustrates a functional configuration of a recovery level estimation device according to a first example embodiment.

FIG. 4 illustrates an example of an eye movement feature.

FIG. 5 is a flowchart of a learning process according to the first example embodiment.

FIG. 6 is a flowchart of a recovery level estimation process according to the first example embodiment.

FIG. 7 illustrates a functional configuration of a recovery level estimation device according to a second example embodiment.

FIG. 8 is a flowchart of a learning process according to the second example embodiment.

FIG. 9 is a flowchart of a recovery level estimation process according to the second example embodiment.

FIG. 10 illustrates a functional configuration of a recovery level estimation device according to a third example embodiment.

FIG. 11 illustrates a specific example of a task.

FIG. 12 is a flowchart of a recovery level estimation process according to a third example embodiment.

FIG. 13 is a block diagram illustrating a functional configuration of a recovery level estimation device according to a fourth example embodiment.

FIG. 14 is a flowchart of a recovery level estimation process according to the fourth example embodiment.

### EXAMPLE EMBODIMENTS

In the following, example embodiments will be described with reference to the accompanying drawings.

### First Example Embodiment

(Configuration)

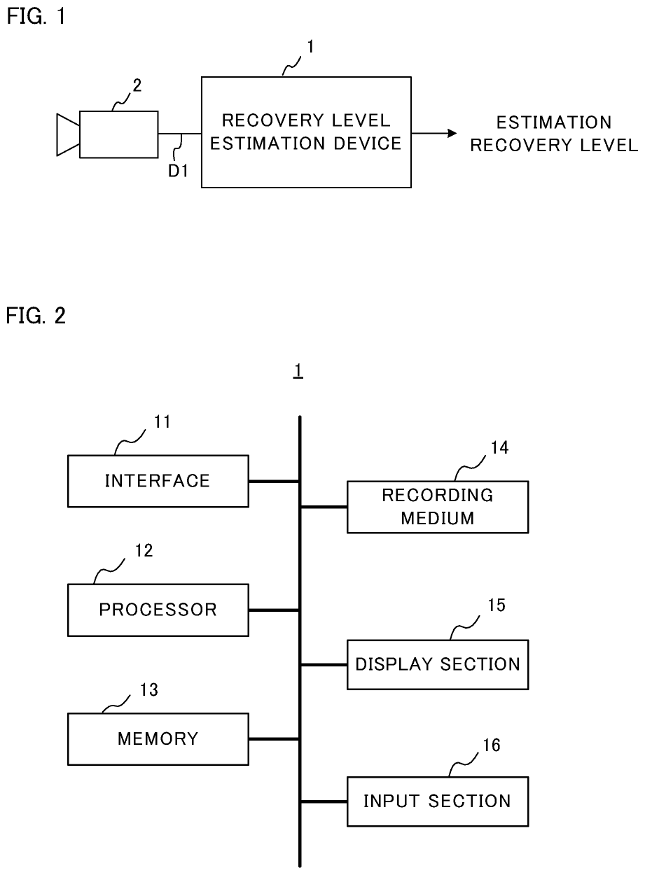

FIG. 1 illustrates a schematic configuration of a recovery level estimation device according to a first example embodiment of the present disclosure. A recovery level estimation device 1 is connected to a camera 2. The camera 2 captures eyes of a patient for whom a recovery level is estimated (hereinafter, simply referred to as the “patient”), and transmits captured images D1 to the recovery level estimation device 1. The camera 2 is assumed to use a high-speed camera capable of capturing images of eyes at a high speed, for instance, 1,000 frames per second. The recovery level estimation device 1 estimates the recovery level by analyzing the captured images D1 and calculating an estimation recovery level.

FIG. 2 is a block diagram illustrating a hardware configuration of the recovery level estimation device 1, As illustrated, the recovery level estimation device 1 includes an interface (interface) 11, a processor 12, a memory 13, a recording medium 14, a display section 15, and an input section 16.

The interface 11 exchanges data with the camera 2. The interface 11 is used when receiving the captured images D1 generated by the camera 2, Moreover, the interface 11 is used when the recovery level estimation device 1 transmits and receives data to and from a predetermined device connected by a wired or wireless communication.

The processor 12 corresponds to one or more processors each being a computer such as a CPU (Central Processing Unit) and controls the whole of the recovery level estimation device 1 by executing programs prepared in advance. The memory 13 is formed by a ROM (Read Only Memory) and a RAM (Random Access Memory). The memory 13 stores the programs executed by the processor 12. Moreover, the memory 13 is used as a working memory during executions of various processes performed by the processor 12.

The recording medium 14 is a non-volatile and non-transitory recording medium such as a disk-shaped recording medium or a semiconductor memory and is formed to be detachable with respect to the recovery level estimation device 1. The recording medium 14 records the various programs executed by the processor 12. When the recovery level estimation device 1 executes a recovery level estimation process, a program recorded in the recording medium 14 is loaded into the memory 13 and executed by the processor 12,

The display section 15 is, for instance, an LCD (Liquid Crystal Display and displays the estimation recovery level or the like which indicates a result of estimating the recovery level of the patient. The display section 15 may display the task of the third example embodiment to be described later. The input section 16 is a keyboard, a mouse, a touch panel, or the like, and is used by an operator such as a medical professional or a specialist.

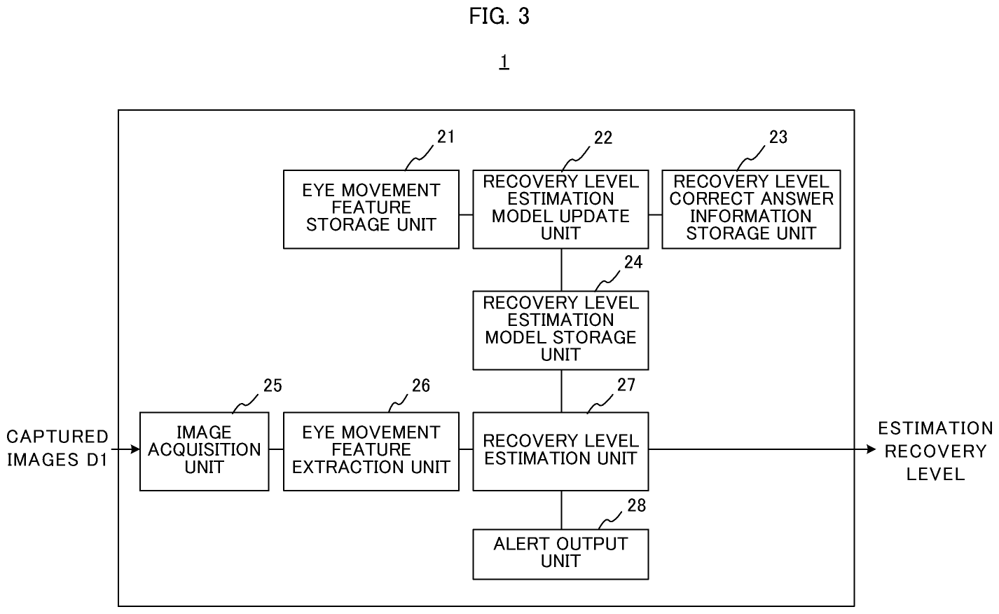

FIG. 3 is a block diagram illustrating a functional configuration of the recovery level estimation device 1. Functionally, the recovery level estimation device 1 includes an eye movement feature storage unit 21, a recovery level estimation model update unit 22, a recovery level correct answer information storage unit 23, a recovery level estimation model storage unit 24, an image acquisition unit 25, an eye movement feature extraction unit 26, a recovery level estimation unit 27, and an alert output unit 28. Note that the recovery level estimation model update unit 22, the image acquisition unit 25, the eye movement feature extraction unit 26, the recovery level estimation unit 27, and the alert output unit 28 are realized by the processor 12 executing respective programs. Moreover, the eye movement feature storage unit 21, the recovery level correct answer information storage unit 23, and the recovery level estimation model storage unit 24 are realized by the memory 13.

The recovery level estimation device 1 generates and updates a recovery level estimation model which learns a relationship between an eye movement feature of the patient and the recovery level by referring to eye movements. In detail, the recovery level estimation device 1, for instance, can be applied to estimate the recovery level by rehabilitation from the sequela caused by cerebral infarction. A learning algorithm may use any machine learning technique such as a neural network, a SVM (Support Vector Machine), a logistic regression (Logistic Regression), or the like. In addition, the recovery level estimation device 1 estimates the recovery level by using the recovery level estimation model to calculate the estimation recovery level of the patient based on the eye movement feature of the patient.

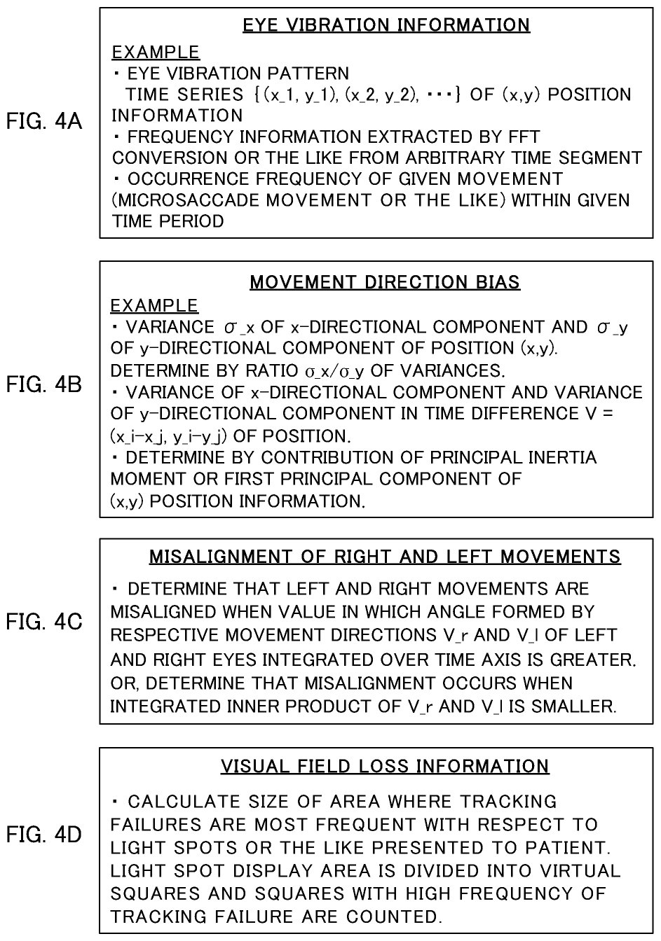

The eye movement feature storage unit 21 stores the eye movement feature used as input data in training of the recovery level estimation model. FIG. 4A to FIG. 4D illustrate examples of the eye movement feature, Each eye movement feature is regarded as a feature of human eye movement, for instance, eye vibration information, a bias of movement directions, a misalignment of right and left movements, visual field defect information, or the like.

As illustrated in FIG. 4A, the eye vibration information is information concerning a vibration of the eyes. Based on the eye vibration information, for instance, abnormalities such as eye tremor and the like caused by the cerebral infarction can be detected. In detail, the eye vibration information may be, for each of a right eye and a left eye, information concerning a time-series change of the coordinates in which, for instance, xy coordinates of the center of a pupil may be taken, or may be frequency information extracted by a FFT (Fast Fourier Transform) or the like within any time segment. Alternatively, the eye vibration information may be information concerning an occurrence frequency within a given time of a predetermined movement such as microsaccard.

As illustrated in FIG. 4B, the bias of the movement direction is regarded as information concerning a bias of movements of the eyes in a vertical direction or a lateral direction. Based on the bias of the movement direction, for instance, it is possible to detect an abnormality such as gaze paralysis or the like caused by the cerebral infarction. In detail, a variance of an x-directional component and a variance of a y-directional component of a position (x, y) are calculated and a ratio of the variances is used to determine the abnormality, or the variance of the x-directional component and the variance of the y-directional component are calculated regarding a time difference of a position of velocity information and a ratio of the variances is used to determine the abnormality, thereby obtaining information on a quantitative bias of the movement directions. Moreover, the bias of the movement directions may be determined and acquired based on a contribution ratio of a principal inertia moment or the first principal component of (x,y) position information.

As illustrated in FIG. 4C, the misalignment of the right and left movements is regarded as information concerning a misalignment of eye movements of the right and left eyes. Based on the misalignment of the right and left movements, for instance, it is possible to detect the abnormality such as strabismus or the like caused by the cerebral infarction. In detail, in a case where an angle between the movement directions of respective right and left eyes is totaled on a time axis, it is determined that the greater the totaled value, the greater the misalignment, or in a case where an inner product of angles formed by respective movement directions of the right and left eyes is totaled on the time axis, it is determined that the smaller the value obtained by totaling the inner products, the greater the misalignment, thereby it is possible to obtain information concerning a quantitative misalignment of the right and left movements.

As illustrated in FIG. 4D, the visual field defect information is information concerning a defect of a visual field. Based on the visual field defect information, for instance, it is possible to detect the abnormality such as gaze failure caused by the cerebral infarction. In detail, the patient is asked to track a light spot being presented and a size of an area where a tracking failure occurs frequently is calculated, or a light spot display area is divided into virtual squares and squares with high frequency of the tracking failure are counted, thereby quantitative visual field loss information can be obtained.

The recovery level correct answer information storage unit 23 stores correct answer information (correct answer label) used in the learning process of training the recovery level estimation model. In detail, the recovery level correct answer information storage unit 23 stores correct answer information for the recovery level for each eye movement feature stored in the eye movement feature storage unit 21. For the recovery level, for instance, a BBS (Berg Balance Scale), a TUG (Timed Up and Go test), a FIM (Functional independence Measure), or the like can be arbitrarily applied.

The recovery level estimation model update unit 22 trains the recovery level estimation model using training data prepared in advance. Here, the training data include the input data and correct answer data. The eye movement feature stored in the eye movement feature storage unit 21 is used as the input data, and the correct answer information for the recovery level stored in the recovery level correct answer information storage unit 23 is used as the correct answer data. In detail, the recovery level estimation model update unit 22 acquires the eye movement feature from the eye movement feature storage unit 21, and acquires the correct answer information for the recovery level corresponding to the eye movement feature from the recovery level correct answer information storage unit 23. Next, the recovery level estimation model update unit 22 calculates the estimation recovery level of the patient based on the acquired eye movement feature by using the recovery level estimation model, and matches the calculated estimation recovery level with the correct answer information for the recovery level, After that, the recovery level estimation model update unit 22 updates the recovery level estimation model to reduce an error between the recovery level calculated by the recovery level estimation model and the correct answer information for the recovery level. The recovery level estimation model update unit 22 overwrites and stores the updated recovery level estimation model in which an estimation accuracy of the recovery level is improved, in the recovery level estimation model storage unit 24.

The recovery level estimation model storage unit 24 stores the updated recovery level estimation model which is trained and updated by the recovery level estimation model update unit 22.

The image acquisition unit 25 acquires the captured images D1 which are obtained by imaging the eyes of the patient and supplied from the camera 2. Note that when the captured images D1 captured by the camera 2 are collected and stored in a database or the like, the image acquisition unit 25 may acquire the captured images D1 from the database or the like.

The eye movement feature extraction unit 26 performs a predetermined image process with respect to the captured images D1 acquired by the image acquisition unit 25, and extracts the eye movement feature of the patient. In detail, the eye movement feature extraction unit 26 extracts time series information of a vibration pattern of the eyes in the captured images D1 as the eye movement feature.

The recovery level estimation unit 27 calculates the estimation recovery level of the patient based on the eye movement feature which the eye movement feature extraction unit 26 extracts, by using the recovery level estimation model. The calculated estimation recovery level is stored in the memory 13 or the like in association with the information concerning the patient.

The alert output unit 28 refers to the memory 13, and outputs the alert to the patient to the display section 15 when the estimation recovery level of the patient deteriorates below a threshold value. In a case where a time period is given for the alert and the estimation recovery level of the patient deteriorates below the threshold value within the given time period, the alert is output.

(Learning Process)

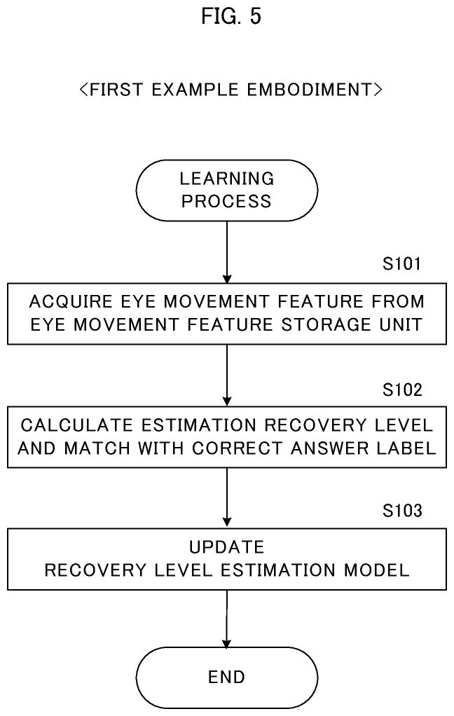

Next, the learning process by the recovery level estimation device 1 will be described, FIG. 5 is a flowchart of the learning process performed by the recovery level estimation device 1. This learning process is realized by executing a program prepared in advance by the processor 12 depicted in FIG. 2.

First, the recovery level estimation device 1 acquires the eye movement feature from the eye movement feature storage unit 21, and acquires the correct answer information for the recovery level with respect to the eye movement feature from the recovery level correct answer information storage unit 23 (step S101), Next, the recovery level estimation device 1 calculates the estimation recovery level based on the acquired eye movement feature by using the recovery level estimation model, and matches the calculated estimation recovery level with the correct answer information for the recovery level (step S102). After that, the recovery level estimation device 1 updates the recovery level estimation model to reduce the error between the estimation recovery level calculated by the recovery level estimation model and the correct answer information for the recovery level (step S103). The recovery level estimation device 1 updates the recovery level estimation model so as to improve the estimation accuracy by repeating this learning process while changing the training data.

(Recovery Level Estimation Process)

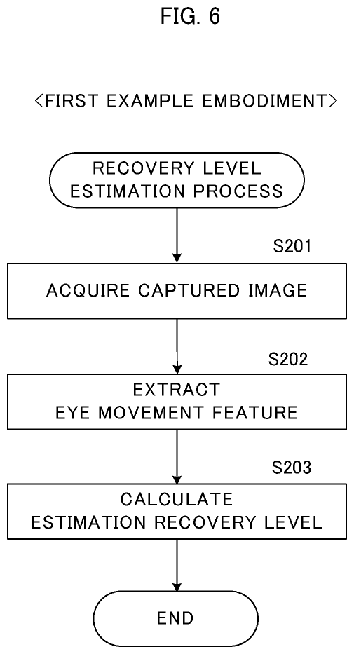

Next, the recovery level estimation process by the recovery level estimation device 1 will be described. FIG. 6 is a flowchart of the recovery level estimation process performed by the recovery level estimation device 1, This recovery level estimation process is realized by executing a program prepared in advance by the processor 12 depicted in FIG. 2.

First, the recovery level estimation device 1 acquires the captured images D1 obtained by capturing the eyes of the patient (step S201), Next, the recovery level estimation device 1 extracts the eye movement feature by an image process from the captured images D1 being acquired (step S202). Next, the recovery level estimation device 1 calculates the estimation recovery level based on the extracted eye movement feature by using the recovery level estimation model (step S203) The estimation recovery level is presented to the patient, the medical professional, and the like in any manner. Accordingly, it is possible for the recovery level estimation device 1 to estimate the recovery level of the patient based on the captured images D1 obtained by capturing the eyes even in an absent of the medical professional or the specialist, and thus it is possible to reduce a burden of the medical professional or the like. Moreover, since a daily recovery level can be predicted even in a sedentary position, the recovery level estimation device 1 can be applied to each patient who has difficulty walking independently without a need for hospital visits or the risk of falling.

Note that the recovery level estimation device 1 stores the calculated estimation recovery level in the memory 13 or the like for each patient, and outputs an alert to the patient to the display section 15 or the like in response to the estimation recovery level of the patient that is worse than the threshold value.

As described above, according to the recovery level estimation device 1 of the first example embodiment, it is possible for the patient to easily and quantitatively measure the estimation recovery level daily at home or elsewhere, and to objectively visualize the daily recovery level and to objectively visualize their daily recovery level. Therefore, it can be expected to increase an amount of rehabilitation due to improved patient motivation for the rehabilitation, and to improve a quality of rehabilitation through frequent revisions of a rehabilitation plan, thereby improving the effectiveness of recovery. In addition, it is possible to detect the abnormality such as a sign of a recurrent cerebral infarction at an early stage, without waiting for an examination or a consultation by the medical professional. Examples of industrial applications of the recovery level estimation device 1 include a remote instruction, a management, and the like of the rehabilitation.

### Second Example Embodiment

(Configuration)

A recovery level estimation device 1x of the second example embodiment utilizes patient information concerning a patient such as an attribute and a recovery record in addition to a eye movement feature, in estimating a recovery level of the patient. Since a schematic configuration and a hardware configuration of the recovery level estimation device 1x are the same as those of the first example embodiment, the explanations thereof will be omitted.

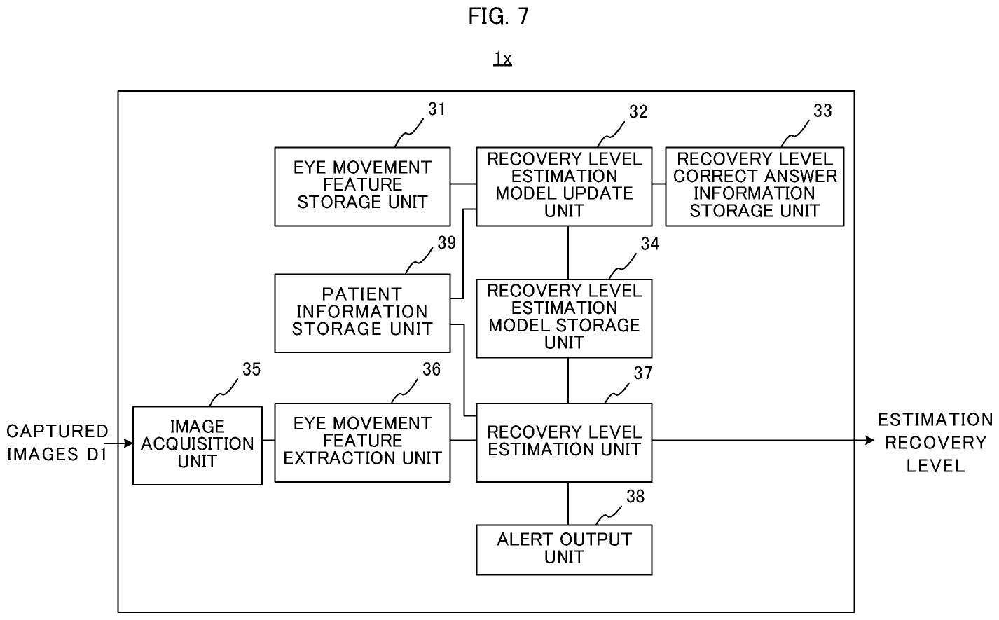

FIG. 7 is a block diagram illustrating a functional configuration of the recovery level estimation device 1x, Functionally, the recovery level estimation device 1x includes an eye movement feature storage unit 31, a recovery level estimation model update unit 32, a recovery level correct answer information storage unit 33, a recovery level estimation model storage unit 34, an image acquisition unit 35, an eye movement feature extraction unit 36, a recovery level estimation unit 37, an alert output unit 38, and a patient information storage unit 39. Note that the recovery level estimation model update unit 32, the image acquisition unit 35, the eye movement feature extraction unit 36, the recovery level estimation unit 37, and the alert output unit 38 are realized by the processor 12 executing respective programs. Also, the eye movement feature storage unit 31, the recovery level correct answer information storage unit 33, the recovery level estimation model storage unit 34, and the patient information storage unit 39 are realized by the memory 13.

The recovery level estimation device 1x of the second example embodiment generates and updates the recovery level estimation model which estimates the recovery level based on the eye movement feature and the patient data of the patient. The learning algorithm may use any machine learning technique such as the neural network, the SVM, the logistic regression, or the like. In addition, the recovery level estimation device 1x calculates the estimation recovery level of the patient by using the recovery level estimation model based on the eye movement feature of the patient and the patient data to estimate the recovery level,

The patient information storage unit 39 stores the patient information concerning the patient. The patient information includes, previous recovery records of the patient including records of attributes such as a gender and an age, a history of the recovery level, a disease name, symptoms, rehabilitation contents, and the like, for instance. The patient information storage unit 39 stores the patient information in association with identification information for each patient.

The recovery level correct answer information storage unit 33 stores the correct answer information for each of respective recovery levels corresponding to combinations of the patient information and the eye movement feature.

The recovery level estimation model update unit 32 trains and updates the recovery level estimation model based on the training data prepared in advance. Here, the training data includes the input data and the correct answer data. In the second example embodiment, the eye movement features stored in the eye movement feature storage unit 31 and the patient information stored in the patient information storage unit 39 are used as the input data. The recovery level correct answer information storage unit 33 stores the correct answer information for the recovery level corresponding to each combination of the eye movement feature and the patient information, and the correct answer information is used as the correct answer data.

In detail, the recovery level estimation model update unit 32 acquires the eye movement feature from the eye movement feature storage unit 31, and acquires the patient information from the patient information storage unit 39. Moreover, the recovery level estimation model update unit 32 acquires the correct answer information for the recovery level corresponding to the acquired patient information and the eye movement feature, from the recovery level correct answer information storage unit 33. Next, the recovery level estimation model update unit 32 calculates the estimation recovery level of the patient based on the eye movement feature and the patient information by using the recovery level estimation model, and matches the estimation recovery level with the correct answer information for the recovery level. After that, the recovery level estimation model update unit 32 updates the recovery level estimation model in order to reduce an error between the recovery level calculated by the recovery level estimation model and the correct answer information for the recovery level. The updated recovery level estimation model is stored in the recovery level estimation model storage unit 34.

The recovery level estimation unit 37 retrieves the patient information of a certain patient from the patient information storage unit 39, and retrieves the eye movement feature of the certain patient from the eye movement feature extraction unit 36. Next, the recovery level estimation unit 37 calculates the estimation recovery level of the certain patient based on the eye movement feature and the patient information by using the recovery level estimation model. The calculated estimation recovery level is stored in the memory 13 or the like in association with the identification information of the certain patient.

Since the eye movement feature storage unit 31, the recovery level estimation model storage unit 34, the image acquisition unit 35, the eye movement feature extraction unit 36, and the alert output unit 38 are the same as in the first example embodiment, the explanations thereof will be omitted.

(Learning Process)

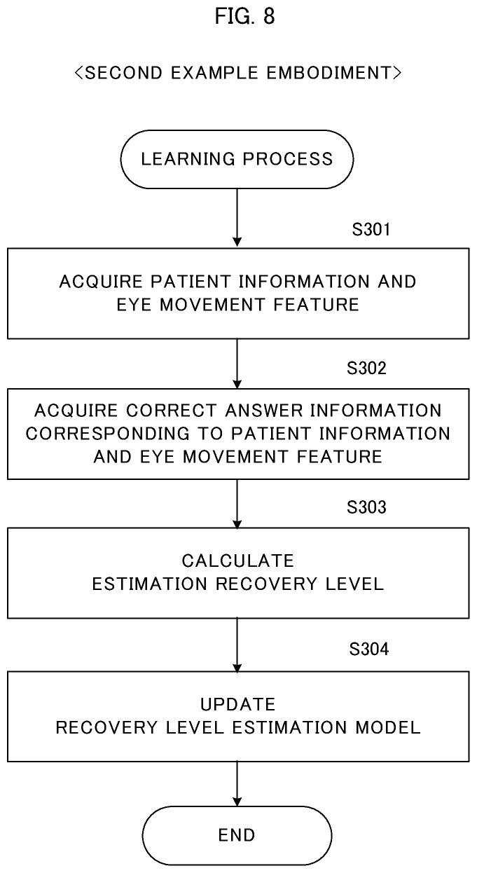

Next, the learning process by the recovery level estimation device 1x will be described. FIG. 8 is a flowchart of the learning process which is performed by the recovery level estimation device 1x. This learning process is realized by executing a program prepared in advance by the processor 12 depicted in FIG. 2.

First, the recovery level estimation device Ix acquires the patient information of a certain patient from the patient information storage unit 39, and acquires the eye movement feature of the patient from the eye movement feature storage unit 31 (step S301). Next, the recovery level estimation device Ix acquires the correct answer information of the recovery level for the patient information and the eye movement feature from the recovery level correct answer information storage unit 33 (step S302). Subsequently, the recovery level estimation device 1x calculates the estimation recovery level of the patient based on the eye movement feature and the patient information, and matches the estimation recovery level with the correct answer information for the recovery level (step S303). After that, the recovery level estimation device 1x updates the recovery level estimation model in order to reduce the error between the estimation recovery level calculated by the recovery level estimation model and the correct answer information of the recovery level (step S304). The recovery level estimation device 1x updates the recovery level estimation model so as to improve the estimation accuracy by repeating the learning process while changing the training data.

(Recovery Level Estimation Process)

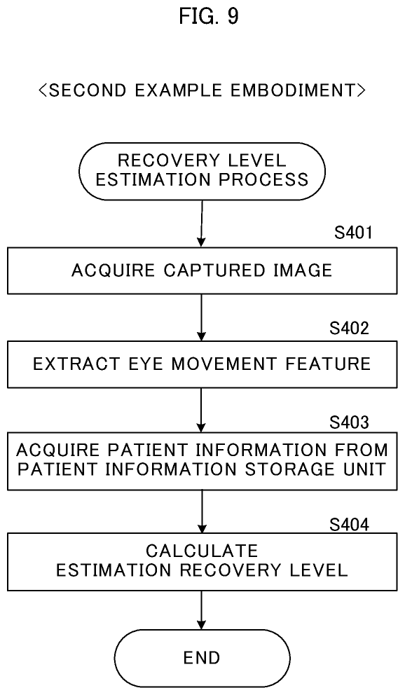

Next, the recovery level estimation process by the recovery level estimation device 1x will be described. FIG. 9 is a flowchart of a recovery level estimation process performed by the recovery level estimation device 1x. This recovery level estimation process is realized by executing a program prepared in advance by the processor 12 depicted in FIG. 2.

First, the recovery level estimation device 1x acquires the captured images D1 obtained by capturing the eyes of the patient (step S401), Next, the recovery level estimation device 1x extracts the eye movement feature from the captured images D1 being acquired, by an imaging process (step S402). Subsequently, the recovery level estimation device 1x acquires the patient information of the patient from the patient information storage unit 39 (step S403). Next, the recovery level estimation device 1x calculates the estimation recovery level of the patient from the extracted eye movement feature and the acquired patient information by using the recovery level estimation model (step S404). After that, the recovery level estimation process is terminated. The estimation recovery level is presented to the patient, the medical professional, or the like in any manner.

Note that the recovery level estimation device 1x stores the calculated recovery level in the memory 13 or the like for each patient, and outputs an alert to the patient to the display section 15 or the like when the estimation recovery level of the patient is worse than the threshold value.

As described above, according to the recovery level estimation device 1x of the second example embodiment, since the recovery level estimation model which estimates the recovery level based on the eye movement feature and the patient information is used, it is possible to estimate the recovery level by considering an individuality and features of each patient.

### Third Example Embodiment

(Configuration)

A recovery level estimation device 1y of a third example embodiment presents a task in capturing eyes of a patient. The task corresponds to a predetermined condition or a task related to the eye movement. By presenting the patient with the task in a case of capturing images of the eyes, the recovery level estimation device 1y is capable of capturing images from which the eye movement feature necessary for estimating the recovery level is easily extracted,

Incidentally, different from the first example embodiment and the second example embodiment, the recovery level estimation device 1y of the third example embodiment internally includes the camera 2. The interface 11, the processor 12, the memory 13, the recording medium 14, the display section 15, and the input section 16 are the same as those of the first example embodiment and the second example embodiment, and explanations thereof will be omitted.

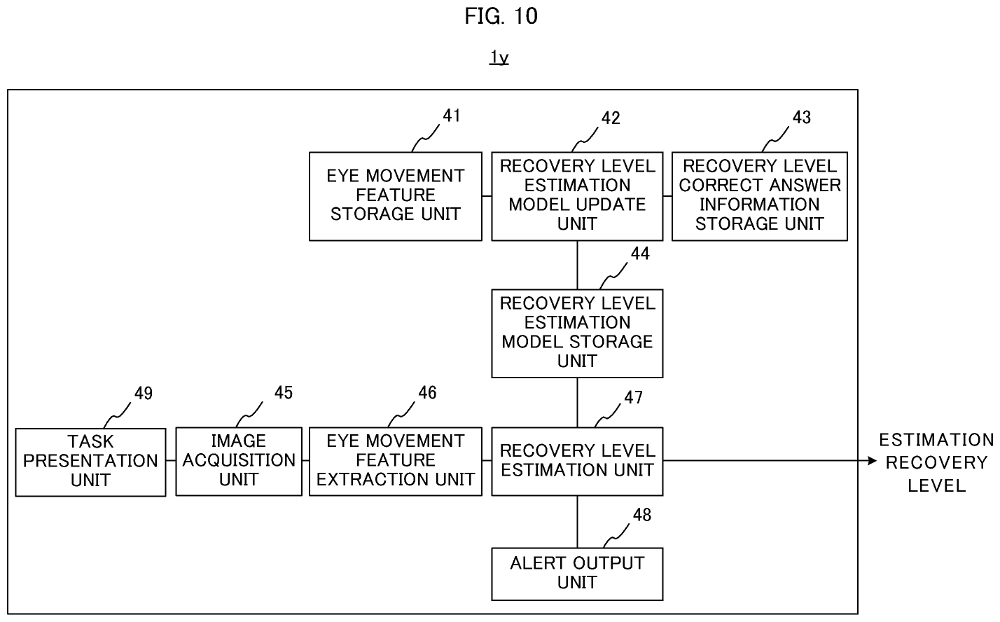

FIG. 10 is a block diagram illustrating a functional configuration of the recovery level estimation device 1y. Functionally, the recovery level estimation device 1y includes an eye movement feature storage unit 41, a recovery level estimation model update unit 42, a recovery level correct answer information storage unit 43, a recovery level estimation model storage unit 44, an image acquisition unit 45, an eye movement feature extraction unit 46, a recovery level estimation unit 47, an alert output unit 48, and a task presentation unit 49. Note that the recovery level estimation model update unit 42, the image acquisition unit 45, the eye movement feature extraction unit 46, the recovery level estimation unit 47, the alert output unit 48, and the task presentation unit 49 are realized by the processor 12 executing respective programs. Moreover, the eye movement feature storage unit 41, the recovery level correct answer information storage unit 43 and the recovery level estimation model storage unit 44 are realized by the memory 13.

By referring to the eye movement, the recovery level estimation device 1y generates and updates the recovery level estimation model which has been trained regarding a relationship between the eye movement feature and the recovery level. The learning algorithm may use any machine learning technique such as the neural network, the SVM, the logistic regression, or the like. Moreover, the recovery level estimation device 1y presents a task concerning the eye movement to the patient, and acquires the captured images D1 which capture the eyes of the patient whom the task has been presented. Accordingly, the recovery level estimation device 1y estimates the recovery level by calculating the estimation recovery level of the patient from the eye movement feature of the patient based on the captured images D1 being acquired, by using the recovery level estimation model.

The task presentation unit 49 presents the task to the patient on the display section 15. The task is a predetermined condition or a task related to the eye movement, and may be arbitrarily set such as “viewing a predetermined image with variation”, “following a moving light spot with the eyes”, or the like, for instance.

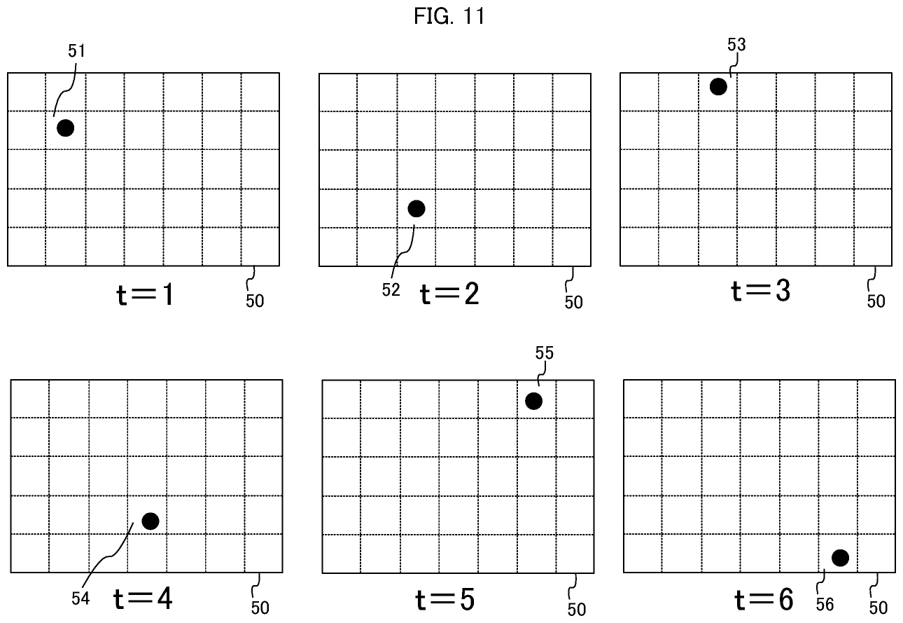

FIG. 11 illustrates a specific example of the task “following a moving light spot with the eyes”. In a light point display region 50 depicted in FIG. 11, a black circle is a light point, and moves to a square 51 at an elapsed time of 1 second (t=1), a square 52 at elapsed time of 2 seconds (t=2), a square 53 at an elapsed time of 3 seconds (t=3), a square 54 at an elapsed time of 4 seconds (t=4), a square 55 at an elapsed time of 5 seconds (t=5), and a square 56 at an elapsed time of 6 seconds (t=6). The patient tracks the moving light spot over time with the eyes of the patient, By presenting the task, the camera 2 built into the recovery level estimation device 1y can easily capture images including the visual field defect information of the patient.

The image acquisition unit 45 acquires the captured images D1 by capturing the eyes moved by the patient along the task with the camera 2 built into the recovery level estimation device.

Note that since the eye movement feature storage unit 41, the recovery level estimation model update unit 42, the recovery level correct answer information storage unit 43, the recovery level estimation model storage unit 44, the eye movement feature extraction unit 46, the recovery level estimation unit 47, and the alert output unit 48 are the same as those in the first example embodiment, the explanations thereof will be omitted. Since the learning process by the recovery level estimation device 1y is the same as that in the first example embodiment, the explanations thereof will be omitted.

(Recovery Level Estimation Process)

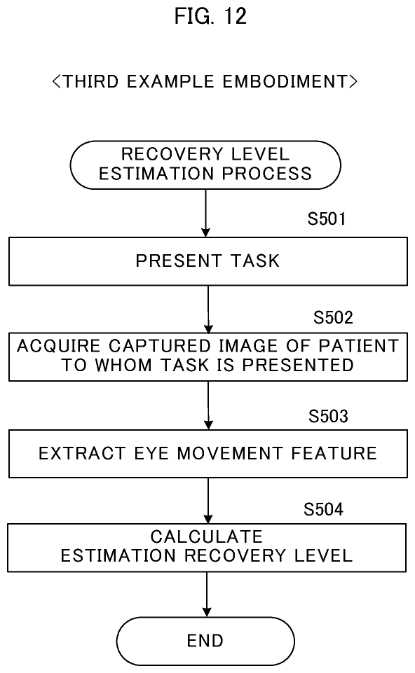

Next, a recovery level estimation process by the recovery level estimation device 1y will be described. FIG. 12 is a flowchart of the recovery level estimation process performed by the recovery level estimation device 1y. This recovery level estimation process is realized by executing a program prepared in advance by the processor 12 depicted in FIG. 2.

First, the recovery level estimation device 1y presents the task to the patient using the display section 15 or the like (step S501). Next, the recovery level estimation device 1y captures the eyes of the patient whom the task is presented, by the camera 2, and acquires the captured images D1 (step S502). In addition, the recovery level estimation device 1y extracts the eye movement feature from the captured images D1 which have been acquired, by the imaging process (step S503). Subsequently, the recovery level estimation device 1y calculates the patient estimation recovery level based on the extracted eye movement feature by using the recovery level estimation model (step S504), The estimation recovery level is presented to the patient, the medical professional, and the like in any manner. By presenting a predetermined task as described above, it is possible for the recovery level estimation device 1y to acquire the captured images D1 from which the eye movement feature is easily extracted.

Note that the recovery level estimation device 1y stores the calculated recovery level in the memory 13 or the like for each patient, and outputs an alert to the patient to the display section 15 or the like in response to the estimation recovery level of the patient that is worse than the threshold value.

Moreover, in the third example embodiment, for convenience of explanations, the recovery level estimation device 1y incorporates the camera 2, and presents the task on the display section 15. However, the present disclosure is not limited thereto, and the recovery level estimation device may internally include the camera 2 and be connected to the camera 2 by a wired or wireless communication to exchange data. In this case, the recovery level estimation device 1y outputs the task for the patient to the camera 2, and acquires the captured images D1 which the camera 2 has been captured.

Moreover, the recovery level estimation device 1y in the third example embodiment may use the patient information, similar to the recovery level estimation model described in the second example embodiment. Furthermore, the recovery level estimation device 1 in the first example embodiment and the recovery level estimation device 1x in the second example embodiment may present the task described in this example embodiment.

### Fourth Example Embodiment

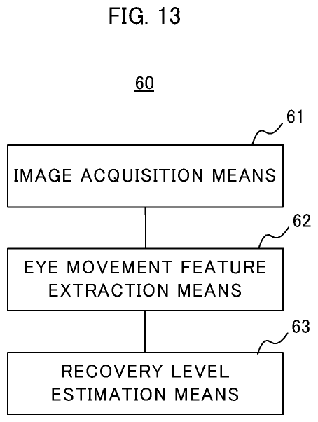

FIG. 13 is a block diagram illustrating a functional configuration of a recovery level estimation device according to a fourth example embodiment. A recovery level estimation device 60 includes an image acquisition means 61, an eye movement feature extraction means 62, and a recovery level estimation means 63.

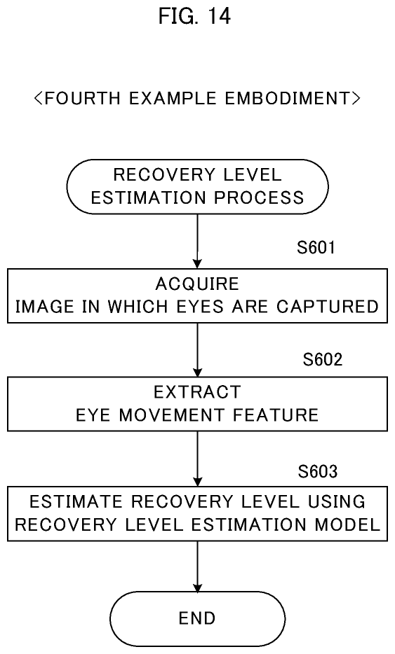

FIG. 14 is a flowchart of a recovery level estimation process performed by the recovery level estimation device 60. The image acquisition means 61 acquires images obtained by capturing the eyes of the patient (step S601). The eye movement feature extraction means 62 extracts the eye movement feature which is a feature of the eye movement based on the images (step S602). The recovery level estimation means 63 estimates the recovery level based on the eye movement feature by using the recovery level estimation model which is learned by machine learning in advance (step S603).

According to the recovery level estimation device 60 of the fourth example embodiment, based on the images obtained by capturing the eyes of the patient, it is possible to estimate the recovery level of the patient with a predetermined disease.

A part or all of the example embodiments described above may also be described as the following supplementary notes, but not limited thereto.

(Supplementary note 1)

A recovery level estimation device comprising:

an image acquisition means configured to acquire images in which eyes of a patient are captured;

an eye movement feature extraction means configured to extract an eye movement feature which is a feature of an eye movement based on the images; and

a recovery level estimation means configured to estimate a recovery level of the patient based on the eye movement feature by using a recovery level estimation model which has been learned by machine learning in advance.

(Supplementary note 2)

The recovery level estimation device according to supplementary note 1, wherein the eye movement feature includes eye vibration information concerning vibrations of the eyes.

(Supplementary note 3)

The recovery level estimation device according to supplementary note 1 or 2, wherein the eye movement feature includes information concerning one or more of a bias of movement directions of the eyes and a misalignment of right and left movements.

(Supplementary note 4)

The recovery level estimation device according to any one of supplementary notes 1 to 3, further comprising a task presentation means configured to present a task concerning eye movements, wherein

the image acquisition means acquires the images of the eyes of the patient whom the task is presented, and

the eye movement feature extraction means extracts the eye movement feature in the task based on the images.

(Supplementary note 5)

The recovery level estimation device according to supplementary note 4, wherein the eye movement feature includes visual field defect information concerning a visual field defect.

(Supplementary note 6)

The recovery level estimation device according to supplementary note 1, further comprising a patient information storage means configured to store patient information concerning one or more of an attribute of the patient and previous recovery records of the patient,

wherein the recovery level estimation means estimates a recovery level of the patient based on the patient information and the eye movement feature.

(Supplementary note 7)

The recovery level estimation device according to supplementary note 1, further comprising an alert output means configured to output an alert in response to the recovery level of the patient that is worse than a threshold value.

(Supplementary note 8)

A method comprising:

acquiring images in which eyes of a patient are captured;

extracting an eye movement feature which is a feature of an eye movement based on the images; and

estimating a recovery level of the patient based on the eye movement feature by using a recovery level estimation model which has been learned by machine learning in advance.

(Supplementary note 9)

A recording medium storing a program, the program causing a computer to perform a process comprising:

acquiring images in which eyes of a patient are captured;

extracting an eye movement feature which is a feature of an eye movement based on the images; and

estimating a recovery level of the patient based on the eye movement feature by using a recovery level estimation model which has been learned by machine learning in advance.

While the disclosure has been described with reference to the example embodiments and examples, the disclosure is not limited to the above example embodiments and examples. It will be understood by those of ordinary skill in the art that various changes in form and details may be made therein without departing from the spirit and scope of the present disclosure as defined by the claims.

### DESCRIPTION OF SYMBOLS

1, 1x, 1y
2 Recovery level estimation device
2 Camera
11 Interface
12 Processor
13 Memory
14 Recording medium
Display section
16 Input section
21, 31, 41 Eye movement feature storage unit
22, 32, 42 Recovery level estimation model update unit
23, 33, 43 Recovery level correct answer information storage unit
24, 34, 44 Recovery level estimation model storage unit
25, 35, 45 Image acquisition unit
26, 36, 46 Eye movement feature extraction unit
27, 37, 47 Recovery level estimation unit
28, 38, 48 Alert output unit
39 Patient information storage unit
49 Task presentation unit

What is claimed is:

1. A recovery level estimation device comprising:
a memory storing instructions; and
one or more processors configured to execute the instructions to:
acquire images in which eyes of a patient are captured;
extract an eye movement feature which is a feature of an eye movement based on the images; and
estimate a recovery level of the patient based on the eye movement feature by using a recovery level estimation model which has been learned by machine learning in advance.

2. The recovery level estimation device according to claim 1, wherein the eye movement feature includes eye vibration information concerning vibrations of the eyes.

3. The recovery level estimation device according to claim 1, wherein the eye movement feature includes information concerning one or more of a bias of movement directions of the eyes and a misalignment of right and left movements.

4. The recovery level estimation device according to claim 1, wherein the processor is further comprising a task presentation moan: configured to present a task concerning eye movements, wherein
the processor acquires the images of the eyes of the patient whom the task is presented, and
the processor extracts the eye movement feature in the task based on the images.

5. The recovery level estimation device according to claim 4, wherein the eye movement feature includes visual field defect information concerning a visual field defect.

6. The recovery level estimation device according to claim 1, wherein the processor is further configured to store patient information concerning one or more of an attribute of the patient and previous recovery records of the patient,
wherein the processor estimates a recovery level of the patient based on the patient information and the eye movement feature.

7. The recovery level estimation device according to claim 1, wherein the processor is further configured to output an alert in response to the recovery level of the patient that is worse than a threshold value.

8. A method comprising:
acquiring images in which eyes of a patient are captured;
extracting an eye movement feature which is a feature of an eye movement based on the images; and
estimating a recovery level of the patient based on the eye movement feature by using a recovery level estimation model which has been learned by machine learning in advance.

9. A non-transitory computer-readable recording medium storing a program, the program causing a computer to perform a process comprising:
acquiring images in which eyes of a patient are captured;
extracting an eye movement feature which is a feature of an eye movement based on the images; and
estimating a recovery level of the patient based on the eye movement feature by using a recovery level estimation model which has been learned by machine learning in advance.

10. The recovery level estimation device according to claim 7, wherein the processor outputs the alert with respect to a medical professional in order for the medical professional to optimize a rehabilitation plan of the patient.
